(19) United States
(12) Patent Application Publication
(10) Pub. No.: US 2005/0251091 A1
(43) Pub. Date: Nov. 10, 2005

(54) Apparatus and methods for transgastric tissue manipulation

(75) Inventors: Vahid Saadat, Saratoga, CA (US); Richard C. Ewers, Fullerton, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI MEDICAL INC., SAN CLEMENTE, CA (US)

(21) Appl. No.: 10/843,682

(22) Filed: May 10, 2004

(51) Int. Cl. A61M 29/00; A61B 1/00; A61B 1/04; A61M 5/178

(52) U.S. Cl. 604/96.01; 600/115; 604/164.03; 604/164.09

(57) ABSTRACT

Apparatus and methods for transgastric tissue manipulation are described herein. Procedures are performed transgastrically through a trocar or insert, e.g., to create tissue plications and approximating those plications towards one another for accomplishing gastroplasty procedures. The trocar is positioned through the abdominal wall of the patient and into the stomach. A multi-lumen insertion tool is positioned within the trocar and comprises one or several channels, e.g., two, three, or more channels as practicable, through the single insertion tool. Each of the channels is aligned and individually sealable with a reversible seal which allows for insertion or removal of a tool therethrough without comprising sealing of the entire trocar or port. A proximal section of the shafts comprise a flexible section to allow for the flexing of each respective control handle away from one another such that the surgeon can manipulate the tools without interference between the handles.

### BACKGROUND OF THE INVENTION

1. Field of the Invention

The present invention relates to apparatus and methods for accessing and manipulating tissue within a hollow body organ. More particularly, the present invention relates to apparatus and methods for transgastrically accessing a hollow body organ for manipulating or otherwise treating the tissue within the hollow body organ and tools for facilitating transgastric access.

2. Background of the Invention

Morbid obesity is a serious medical condition pervasive in the United States and other countries. Its complications include hypertension, diabetes, coronary artery disease, stroke, congestive heart failure, multiple orthopedic problems and pulmonary insufficiency with markedly decreased life expectancy.

A number of surgical techniques have been developed to treat morbid obesity, e.g., bypassing an absorptive surface of the small intestine, or reducing the stomach size. However, many conventional surgical procedures may present numerous life-threatening post-operative complications, and may cause atypical diarrhea, electrolytic imbalance, unpredictable weight loss and reflux of nutritious chyme proximal to the site of the anastomosis.

Furthermore, the sutures or staples that are often used in these surgical procedures typically require extensive training by the clinician to achieve competent use, and may concentrate significant force over a small surface area of the tissue, thereby potentially causing the suture or staple to tear through the tissue. Many of the surgical procedures require regions of tissue within the body to be approximated towards one another and reliably secured. The gastrointestinal lumen includes four tissue layers, wherein the mucosa layer is the inner-most tissue layer followed by connective tissue, the muscularis layer and the serosa layer.

One problem with conventional gastrointestinal reduction systems is that the anchors (or staples) should engage at least the muscularis tissue layer in order to provide a proper foundation. In other words, the mucosa and connective tissue layers typically are not strong enough to sustain the tensile loads imposed by normal movement of the stomach wall during ingestion and processing of food. In particular, these layers tend to stretch elastically rather than firmly hold the anchors (or staples) in position, and accordingly, the more rigid muscularis and/or serosa layer should ideally be engaged. This problem of capturing the muscularis or serosa layers becomes particularly acute where it is desired to place an anchor or other apparatus transesophageally rather than intraoperatively, since care must be taken in piercing the tough stomach wall not to inadvertently puncture adjacent tissue or organs.

One conventional method for securing anchors within a body lumen to the tissue is to utilize sewing devices to suture the stomach wall into folds. This procedure typically involves advancing a sewing instrument through the working channel of an endoscope and into the stomach and against the stomach wall tissue. The contacted tissue is then typically drawn into the sewing instrument where one or more sutures or tags are implanted to hold the suctioned tissue in a folded condition known as a plication. Another method involves manually creating sutures for securing the plication.

One of the problems associated with these types of procedures is the time and number of intubations needed to perform the various procedures endoscopically. Another problem is the time required to complete a plication from the surrounding tissue with the body lumen. In the period of time that a patient is anesthetized, procedures such as for the treatment of morbid obesity or for GERD must be performed to completion. Accordingly, the placement and securement of the tissue plication should ideally be relatively quick and performed with a minimal level of confidence.

Another problem is obtaining access within the patient to the tissue to be plicated or manipulated. Access to the tissue, e.g., the stomach, is conventionally done through various methods. One method is open surgery but this is a highly invasive procedure and often involves a high degree of morbidity. Moreover, open surgical procedures typically increase the healing time necessary for the patient and also increases the degree of pain and leaves relatively large scars on the patient.

Other methods involve laparoscopic procedures in which multiple laparotomies are made in a patient through which rigid elongate tools are inserted into the patient. However, laparoscopic procedures are lengthy, technically demanding, and require multiple incisions in a patient at various locations to provide for tool access to the tissue.

### BRIEF SUMMARY OF THE INVENTION

Procedures which are suited for performing transgastrically through a trocar or insert described below involve creation of tissue plications. These plications may also be approximated towards one another transgastrically for accomplishing gastroplasty procedures.

To facilitate such transgastric procedures in a patient, a transgastric assembly may generally comprise a conventional trocar or laparoscopic port which may be positioned through the abdominal wall of the patient and through a gastrostomy into a hollow body organ such as the stomach. The trocar or port may be positioned anteriorly of the greater curvature of the stomach, e.g., within the region of the antrum of stomach, to provide tool access to the stomach interior, particularly to tissue around the lesser curvature. Other procedures may utilize trocars or ports positioned to access the peritoneal cavity or other hollow body organs.

A multi-lumen insertion tool may be positioned within the trocar during or after placement of trocar within the stomach and generally comprises one or several channels or passageways, e.g., two, three, or more channels as practicable, through a single insertion tool. Alternatively, the body of the multi-lumen insertion tool itself may be articulatable independently of the tools inserted therethrough. In other variations, a conventional endoscopic device may alternatively be inserted through the trocar and the tools may be advanced through the endoscope towards the tissue region of interest. Having the multiple distinct lumens defined through a singular access device enables the passage of multiple tools through a single access trocar or port.

Each of the channels is preferably aligned and individually sealable with a reversible seal which allows for the insertion or removal of a tool therethrough without comprising sealing of the entire trocar or port. Moreover, because each channel is independently sealed, individual tools may be inserted and/or removed from the insertion tool without disturbing the orientation and/or seal of adjacent tools. The trocar or port may also comprise at least one inflatable or expandable element, e.g., inflatable balloon, which may be disposed along an outer surface of the trocar or port for inflation against the stomach tissue. Optionally, an additional inflatable balloon may be provided for inflation on a proximal region of the trocar for inflation against the patient's skin surface.

Any number of tools may be advanced through the trocar and depending upon the desired procedure to be performed upon the tissue, each tool may have an appropriate end effector located on or near its distal end. For example, a tissue plication assembly disposed at the distal end of an elongate tool shaft and/or a tissue manipulation assembly disposed at the distal end of an elongate tool shaft may be inserted through the trocar for tissue plication and approximation procedures to be performed upon the tissue. One or both of the elongate tool shafts may be rigid such that each of shafts distal of the trocar maintains a parallel orientation with respect to one another.

A proximal section of one or both shafts may each comprise flexible sections to allow for the flexing of each respective control handle away from one another such that the surgeon or user may freely manipulate the assemblies without interference between the control handles. Moreover, each of the flexible sections is preferably configured to allow for torquing forces to be transmitted over the lengths of the flexible section as well as to allow for longitudinal forces to be transmitted along the length of shafts so that the shafts may be translated longitudinally through the trocar.

To provide imaging of the tools and tissue during a procedure, an imaging device such as in an endoscope or laparoscope may be advanced through one of the lumens in the insertion tool. Alternatively, an endoscope may be advanced transesophageally through the patient's esophagus and its distal end may be retroflexed and oriented towards the tissue region of interest to provide not only imaging, but also light, delivery of fluids, etc., as desired.

In other alternatives, additional trocars may be positioned into the stomach to provide access paths for additional tools or imaging devices. The additional trocars may or may not utilize the multi-lumen insertion tool.

### BRIEF DESCRIPTION OF THE DRAWINGS

FIG. 1 shows an example of multiple tools advanced transgastrically into the stomach through a single port or trocar for accessing and manipulating the tissue within.

FIG. 2A shows a single port or trocar in place within the stomach and an example of the surgical arc available for tool access.

FIG. 2B shows a variation of a port or trocar having two inflatable members for securing its position transgastrically.

FIG. 3 shows a detail view of a port or trocar variation in partial cross-section with an optional multi-lumen insertion tool positionable within the port or trocar.

FIGS. 4A and 4B show perspective and end views of alternative multi-lumen insertion tools having two or more insertion channels.

FIG. 4C shows a perspective view of another multi-lumen insertion tool variation having an articulatable body.

FIG. 5A shows a detail view of a tool having a flexible section along its shaft.

FIG. 5B illustrates a side view of the tool of FIG. 5A showing the possible range of motion for the proximal end of the tool with the flexible section.

FIG. 6 shows one example of how tools advanced transgastrically may be utilized to perform gastroplasty procedures on the tissue.

FIG. 7 shows another example of tools being positioned through an endoscope which may be advanced through the port or trocar for obtaining localized access to the tissue.

FIG. 8 shows yet another example of additional ports or trocars being utilized along with the multi-lumen insertion tool for use of additional tools within the stomach.

FIG. 9 shows an example of how the articulatable multi-lumen insertion tool shown in FIG. 4C may be utilized for a procedure.

### DETAILED DESCRIPTION OF THE INVENTION

Generally, access to the interior of a hollow body organ for manipulation of the tissue may be accomplished through a trocar, port, or other insert. This trocar, port, or insert may be positioned through a small incision on the patient's abdomen and through a gastrostomy made through the patient's stomach for enabling access therethrough with multiple tools preferably via a single access path.

One of the applications for creating a single transgastric access path through which multiple tools may be advanced is in manipulating the tissue and, e.g., creating tissue plications, from within the hollow body organ. Tissue plications may be formed using various tools for approximating tissue in performing various gastroplasty procedures, e.g., for use in treating morbid obesity.

In creating tissue plications, a tissue plication tool having a distal tip may be advanced through the trocar and into the stomach. The tissue may be engaged or grasped and the engaged tissue may be moved to a proximal position relative to the tip of the device, thereby providing a substantially uniform plication of predetermined size. Some examples of the various tools which may be advanced transgastrically through the insertion lumen of the port or trocar described herein are disclosed in U.S. patent application Ser. No. 10/735,030 filed Dec. 12, 2003, which is incorporated herein by reference in its entirety.

Various tools for endoluminally visualizing, grasping, plicating, manipulating, affixing, securing, etc., portions of gastric tissue may be utilized with the insertion lumen for transgastrically performing some or all of these procedures. Other examples of applicable tools may be seen in U.S. patent application Ser. Nos. 10/734,547 and 10/734,562, both filed Dec. 12, 2003 and both incorporated herein by reference in their entirety. Other examples of various tools which may be utilized transgastrically with the trocar described herein are also further described in U.S. patent application Ser. No. 10/639,162 filed Aug. 11, 2003 and Ser. No. 10/672,375 filed Sep. 26, 2003, each of which is also incorporated herein by reference in its entirety.

One particular procedure which is suited for performing transgastrically with the trocar or insert described herein is the creation of tissue plications which may be approximated towards one another for performing gastroplasty procedures, as described in any of the above disclosures which have been incorporated herein by reference. Generally, formation of a tissue fold may be accomplished using at least two tissue contact areas that are separated by a linear or curvilinear distance, wherein the separation distance between the tissue contact points affects the length and/or depth of the fold. In operation, a tissue grabbing assembly engages or grasps the tissue wall in its normal state (i.e., non-folded and substantially flat), thus providing a first tissue contact area. The first tissue contact area then is moved to a position proximal of a second tissue contact area to form the tissue fold. The tissue anchor assembly then may be extended across the tissue fold at the second tissue contact area. Optionally, a third tissue contact point may be established such that, upon formation of the tissue fold, the second and third tissue contact areas are disposed on opposing sides of the tissue fold, thereby providing backside stabilization during extension of the anchor assembly across the tissue fold from the second tissue contact area.

The first tissue contact area may be utilized to engage and then stretch or rotate the tissue wall over the second tissue contact area to form the tissue fold. The tissue fold may then be articulated to a position where a portion of the tissue fold overlies the second tissue contact area at an orientation that is substantially normal to the tissue fold. A tissue anchor may then be delivered across the tissue fold at or near the second tissue contact area. An apparatus in particular which is particularly suited to deliver the anchoring and securement devices described herein may be seen in further detail in co-pending U.S. patent application Ser. No. 10/735,030 filed Dec. 12, 2003, which has been incorporated herein by reference above.

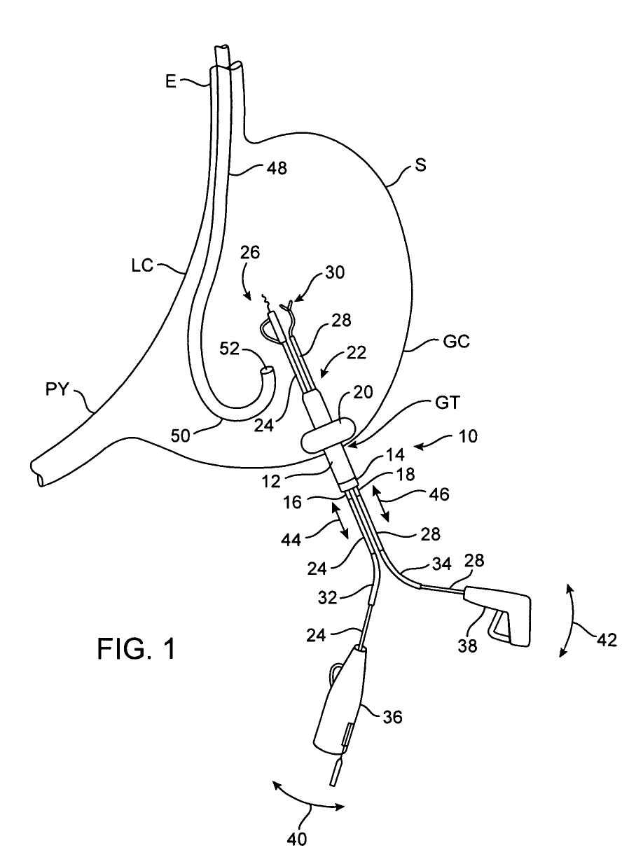

Turning now to the figures, FIG. 1 shows a partial cross-sectional view of transgastric assembly 10 which has been positioned through a gastrostomy to extend partially within stomach S. As seen, transgastric assembly 10 may generally comprise a conventional trocar or laparoscopic port 12 which may be positioned through the abdominal wall of the patient and through gastrostomy GT into a hollow body organ, in this example stomach S. Trocar or port 12 may be positioned anteriorly of the greater curvature GC of the stomach S, e.g., within the region of the antrum of stomach S, to provide tool access to the stomach interior, particularly to tissue around the lesser curvature LC. The pylorus PY is also shown for orientation. Other procedures may utilize trocars or ports positioned to access the peritoneal cavity or other hollow body organs.

Multi-lumen insertion tool 14 may be positioned within trocar 12 during or after placement of trocar 12 within stomach S. Insertion tool 14 may generally comprise one or several channels or passageways, in this example two channels 16, 18, defined through a single insertion tool 14 to thereby enable the passage of multiple tools through a single access trocar or port 12. Each of the channels 16, 18 are preferably aligned parallel with one another and each channel may be individually sealable with a reversible seal which allows for the insertion or removal of a tool therethrough without comprising sealing of the trocar or port 12. Multi-lumen insertion tool 14 may be fabricated from any variety of biocompatible materials, e.g., stainless steel, plastics, etc. Having a reversible seal may help to prevent leakage of any fluids or gases through the trocar or port 12 and/or individual channels 16, 18 during a procedure. Moreover, because each channel 16, 18 may be independently sealable, individual tools may be inserted and/or removed from the insertion tool 14 without disturbing the orientation and/or seal of adjacent tools. Alternatively, a trocar or port having an integrated multi-lumen tool may be fabricated to provide a singular device in which case the trocar and multi-lumen tool may be made from the same material.

Trocar or port 12 may also comprise at least one inflatable or expandable element, e.g., inflatable balloon 20, which may be disposed along an outer surface of trocar or port 12. Inflatable balloon 20 may be inflated within stomach S once trocar 12 has been partially advanced within and then held against the stomach interior surface to provide stability for trocar 12. Trocar 12 may further define an opening 22 through which elongate tool shafts 24, 28 may be advanced for treatment and/or manipulation of the interior stomach tissue.

As described above, any number of tools may be advanced through trocar 12. This particular example illustrates tissue plication assembly 26 disposed at the distal end of elongate tool shaft 24 and tissue manipulation assembly 30 disposed at the distal end of elongate tool shaft 28, although other tools may also be utilized depending upon the desired procedure to be performed upon the tissue. To facilitate advancement of the tool assemblies through trocar 12 and treatment of the tissue, one or both of elongate tool shafts 24, 28 may be rigid such that each of the shafts 24, 28 may maintain a parallel orientation with respect to one another.

However, a proximal section of one or both shafts 24, 28 may each comprise flexible sections 32, 34, respectively, which may allow for the flexing of each respective handle away from one another such that the surgeon or user may freely manipulate the assemblies within stomach S without interference from each handle. As shown in the example, plicator handle 36 may be flexed via flexible section 32 in the direction of arrow 40 while manipulation handle 38 may be flexed via flexible section 34 in the direction of arrow 42. Either or both shafts 24, 28 may utilize a flexible section as desired. Moreover, each of the flexible sections 32, 34 is preferably configured to allow for torquing forces to be transmitted over the lengths of the flexible section as well as to allow for longitudinal forces to be transmitted along the length of shafts 24, 28 such that the shafts may be translated longitudinally in the direction of arrows 44, 46, respectively, through trocar 12.

To provide imaging of the tools and tissue during a procedure, one method may utilize an endoscope 48 advanced transesophageally through the patient's esophagus E such that its distal end may be retroflexed via flexible section 50. An imaging system 52 at the distal end of endoscope 48 may thus be oriented towards the tissue region of interest to provide not only imaging, but also light, delivery of fluids, etc., as desired.

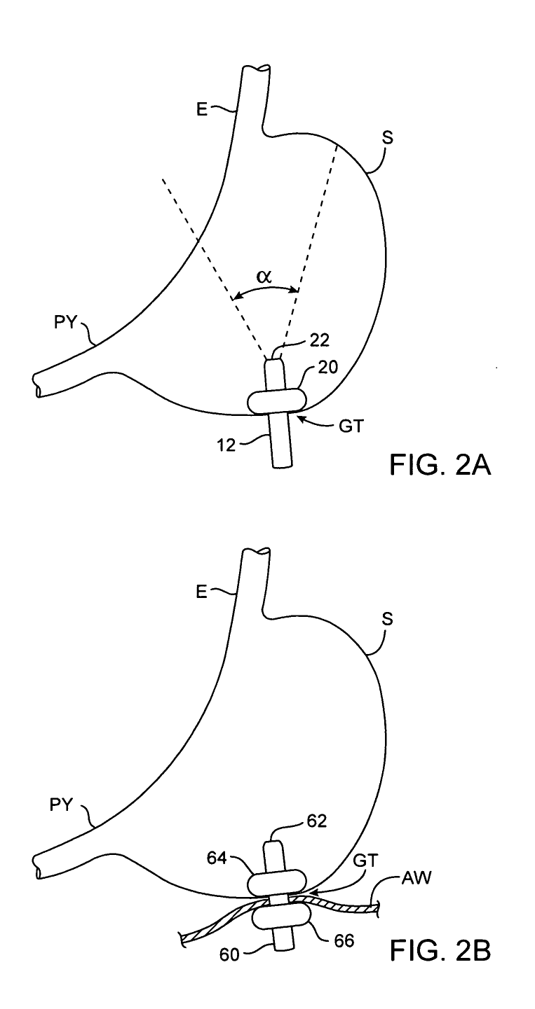

FIG. 2A shows trocar 12 positioned through gastrostomy GT. Although trocar 12 may be rotated or pivoted relative to the surrounding tissue even after trocar 12 has been positioned with inflatable balloon 20, trocar 12 is preferably positioned relative to the stomach such that the tissue region to be treated falls within the surgical arc α, which is conical in shape and represents the region accessible by tools inserted through trocar 12. The surgical arc α may range from 45° to 90°, or even higher depending upon the design of trocar 12 and the degree of pivoting of trocar 12 relative to the surrounding tissue.

FIG. 2B shows an alternative trocar or port 60 defining lumen 62 therethrough and which may comprise not only a distal inflatable member 64 but also a proximal inflatable member 66. Proximal inflatable member 66 may be positioned over a surface of trocar 60 for inflation against the skin surface of abdominal wall AW. Having both distal inflatable member 64 and proximal inflatable member 66 may allow for securement of trocar 12 by enable the sandwiching of abdominal wall AW and stomach S between each of the members 64, 66.

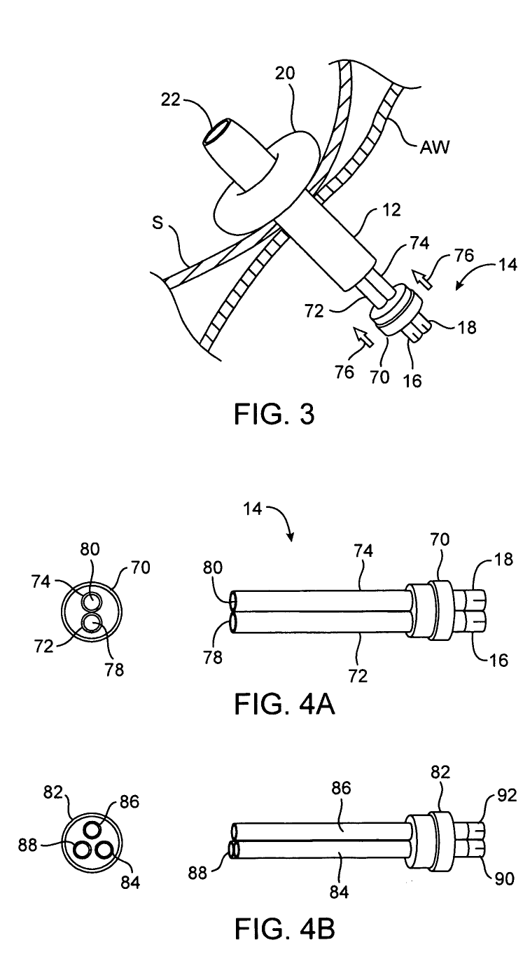

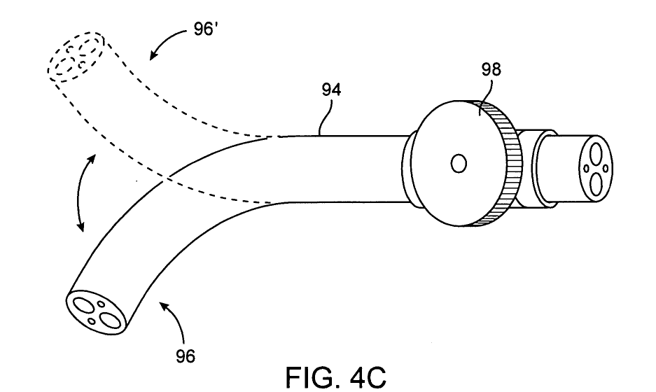

FIG. 3 shows a partial cross-sectional perspective view of trocar 12 with multi-lumen insertion tool 14 partially positioned within trocar 12. The use of insertion tool 14 may be optional and trocar 12 may be utilized without multi-lumen insertion tool 14 for advancing several tools therethrough. FIG. 4A shows a perspective view of insertion tool 14 alone for clarity. As illustrated and as described above, insertion tool 14 may comprise first channel 72 and second channel 74 adjacent, and preferably parallel, to one another. First channel 72 and second channel 74 may each define first lumen 78 and second lumen 80, respectively, for the passage of the various tools therethrough. The lengths of first channel 72 and second channel 74 may be equal or they may be varied; moreover, the lengths may be sufficiently long enough so as to extend distally beyond, coextensively with, or proximally of trocar opening 22.

An insert seal 70 may be positioned along the lengths of first channel 72 and second channel 74 and preferably near a proximal portion of the lengths. Insert seal 70 may be used to provide for a fluid tight seal between insertion tool 14 and trocar 12 as well as to provide for stability between insertion tool 14 and trocar 12. The end view of FIG. 4A shows the orientation of first channel 72 and second channel 74 relative to one another. Insertion tool 14 may be inserted within trocar 12 in the direction as shown by arrows 76 in FIG. 3. Moreover, as mentioned above, each of first channel 72 and second channel 74 may be individually sealed via seals 16, 18, respectively, so as to enable the introduction or removal of an individual tool from insertion tool 14 without disturbing the seal or orientation of adjacent tools.

FIG. 4B shows a perspective view of an alternative variation for the insertion tool having first, second and third channels 84, 86, 88, respectively, with a common insertion seal 82. As above, each of the individual channels 84, 86, 88 may be individually sealed, as shown by seals 90 and 92 (third seal is hidden from view). The end view of FIG. 4B shows a possible orientation of first, second and third channels 84, 86, 88, respectively, with respect to one another.

FIG. 4C shows a perspective view of yet another alternative variation for the insertion tool. As shown, the insertion tool may have an articulatable body 94 for controlling its position independently of the tools inserted therethrough and independently of the trocar or port through which the body 94 may be inserted. The articulatable body 94 may be controlled via a proximally located control 98 for articulating the body 94 between a first position 96 and a second position 96′. The positions and configurations are shown merely for illustrative purposes and are not intended to limit the range of possible motion of the articulatable body 94. The option of having an articulatable body 94 may facilitate fine motion of the assembly for positioning or manipulation of the tools inserted therethrough.

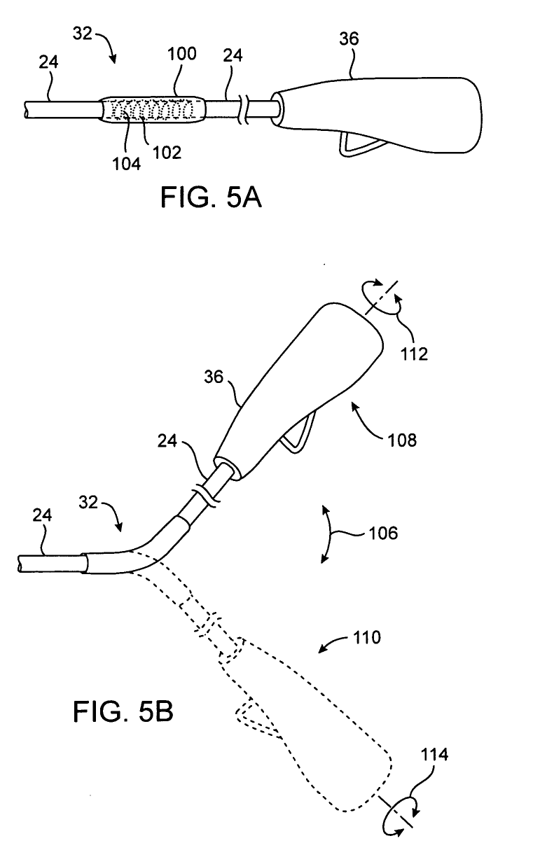

As mentioned above, the proximal sections of one or both elongate tool shafts may be configured to flex relative to one another and to the assembly. FIG. 5A shows a side view of one example of a possible configuration for the flexible coupling section 32. The coupler may comprise a covering 100 preferably made of a fluid-impervious material which is flexible, e.g., rubber, elastomeric materials, etc. Underlying the covering 100 may be a flexible body such as a spring body or a cut section having a plurality of circumferentially defined slits. Coupling section 32 may define lumen 104 therethrough for the passage of control wires, cables, fibers, etc., through the elongate shaft body 24.

The construction of coupling section 32 is preferably such that handle 36 may be flexed rotationally in-plane as well as out-of-plane, as represented by arrow 106 in FIG. 5B. The construction is also such that torque may be transmitted from the handle 36 over coupling section 32 and to the distal end of shaft 24 regardless of the angular orientation of handle 36 with respect to elongate shaft 24, as represented by torque direction 112 of handle 36 in first position 108 and torque direction 114 of handle 36 in second position 110.

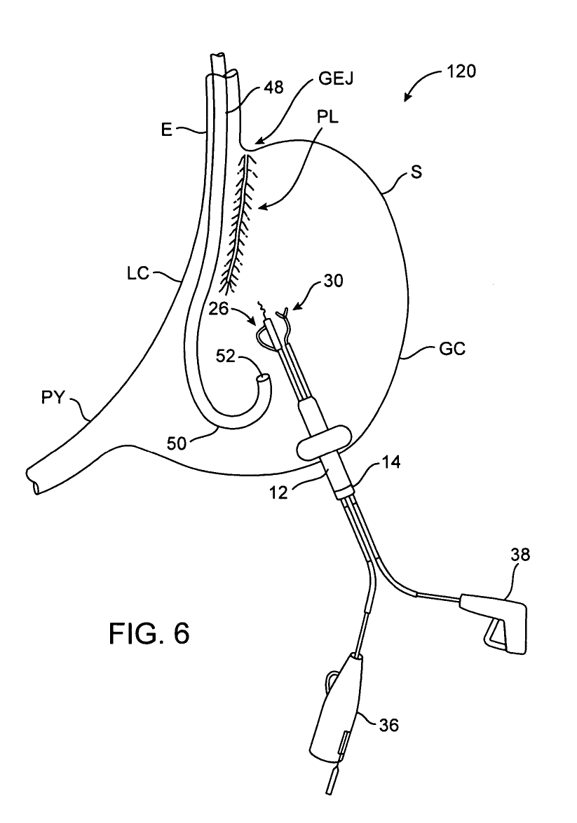

FIG. 6 shows one application of the numerous applications for the transgastric tools and methods described herein. As shown, the transgastric assembly 120 may be used to create a tissue plication PL extending from the gastroesophageal junction GEJ towards the pylorus PY along the lesser curvature LC. Examples for creating tissue plications using a tissue plication assembly 26 is disclosed in detail in U.S. Patent application Ser. No. 10/735,030 filed Dec. 12, 2003, which has been incorporated by reference above.

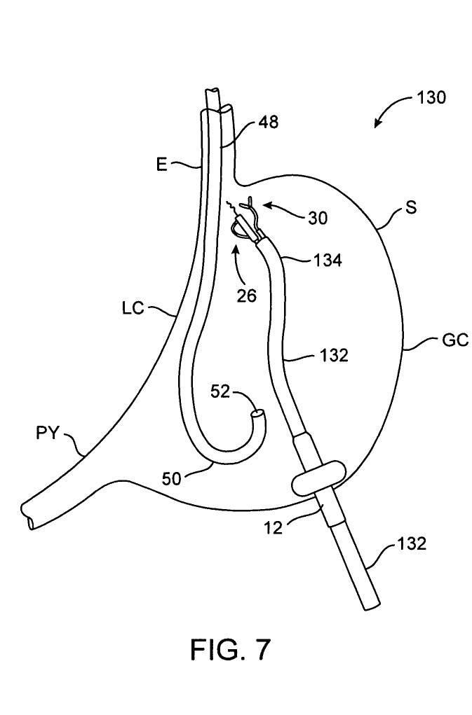

FIG. 7 shows another application in transgastric assembly 130 in which endoscope 132, which may comprise a conventional endoscope or any one of a number of conventional steerable endoscopes, may be advanced through trocar 12 and maneuvered via steerable section 134 into proximity of the tissue to be manipulated.

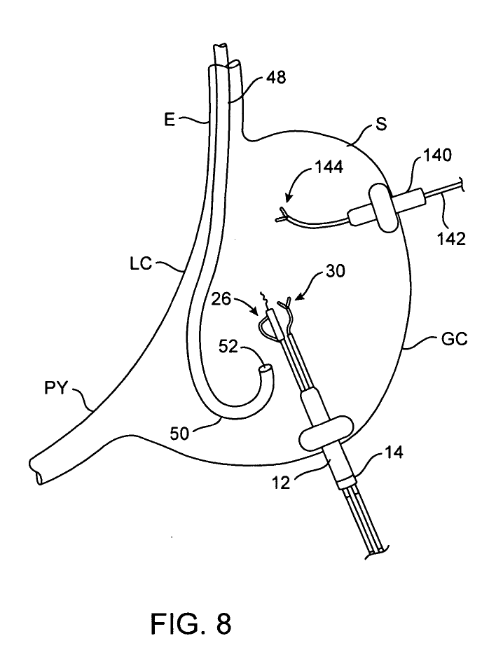

FIG. 8 shows yet another application in which an additional trocar 140 may be positioned into stomach S relative to trocar 12. Additional tools may be advanced through trocar 140 for added capabilities in addition to the tools advanced through trocar 12. The example illustrates tissue manipulator or grasper 144 disposed upon shaft 142 being advanced within stomach S; however, any number of tools or imaging systems may be utilized and any number of additional trocars or ports may also be optionally utilized as practicable, if desired.

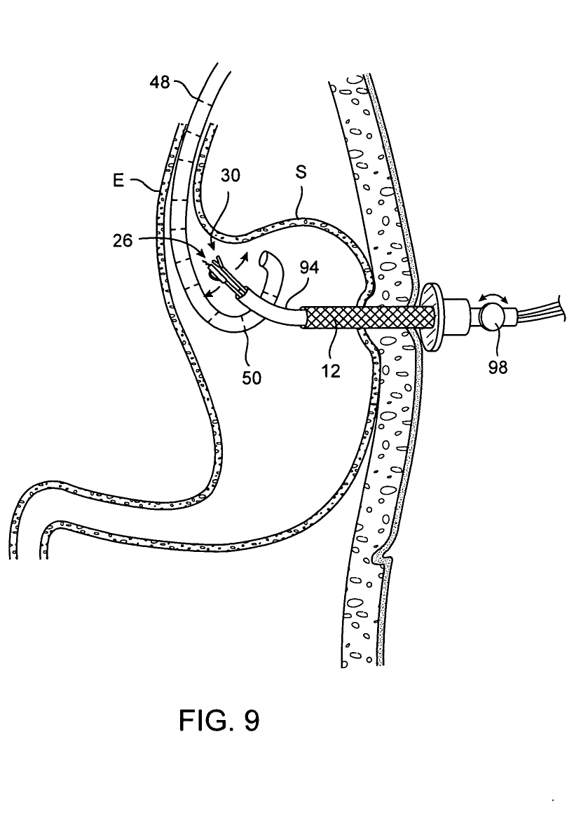

FIG. 9 shows an example of how the articulatable multi-lumen insertion tool shown in FIG. 4C may be utilized for a procedure. As shown, articulatable body 94 may be positioned through trocar 12. The distal portion of body 94 may be advanced distally beyond trocar 12 such that the body 94 may be articulated without constraint from the trocar 12. Articulation of the body 94 is shown for facilitating fine motion positioning of the tools 26, 30 through the trocar 12. Alternatively, articulatable body 94 may be articulated to facilitate tissue manipulation relative to trocar 12.

Although a number of illustrative variations are described above, it will be apparent to those skilled in the art that various changes and modifications may be made thereto without departing from the scope of the invention. Moreover, although specific tools and trocar configurations may be shown, it is intended that the tool, trocar, etc., configurations be utilized with the various types of procedures in various combinations as practicable. It is intended in the appended claims to cover all such changes and modifications that fall within the true spirit and scope of the invention.

1. A system for providing transgastric access, comprising:
an insertion tool having at least two channels defined therethrough for placement within a port; and
an elongate tool member having an end effector and being adapted for insertion through at least one of the channels, wherein a proximal section of the tool member comprises a flexible portion.

2. The system of claim 1 wherein the insertion tool comprises a common seal for sealing against the port.

3. The system of claim 1 wherein each of the channels comprise a seal independent of adjacent channels.

4. The system of claim 1 wherein the insertion tool has at least three channels defined therethrough.

5. The system of claim 1 wherein each of the channels are parallel with one another.

6. The system of claim 1 wherein the insertion tool is integral with the port.

7. The system of claim 1 wherein the insertion tool comprises an articulatable body.

8. The system of claim 1 wherein the elongate tool member has an end effector adapted for tissue plication.

9. The system of claim 1 wherein the elongate tool member has an end effector adapted for tissue approximation.

10. The system of claim 1 wherein the elongate tool member comprises a rigid shaft distal of the flexible portion.

11. The system of claim 1 wherein the port comprises at least one inflatable member over its outer surface.

12. The system of claim 1 further comprising an additional insertion tool for insertion through an additional port.

13. The system of claim 1 wherein the insertion tool is adapted for insertion within a stomach of a patient.

14. A multi-lumen access tool for providing transgastric access, comprising:
at least two channels aligned adjacently and adapted for placement through a single access port;
a common seal disposed about each of the channels and adapted for sealing against the access port; and
an individual seal within each of the channels adapted for sealing a respective channel.

15. The tool of claim 14 further comprising an access port adapted for transgastric placement into a stomach of the patient and defining a single lumen through which the channels are positioned.

16. The tool of claim 14 wherein each of the channels are parallel with respect to one another.

17. The tool of claim 14 wherein each of the channels is integral with the access port.

18. The tool of claim 14 wherein the channels are articulatable relative to the access port.

19. The tool of claim 14 further comprising at least one inflatable member positioned about the channels for securing the access tool to a patient.

20. A method of transgastrically performing a procedure upon tissue within a stomach, comprising:
advancing an access port transgastrically into the stomach of a patient;
advancing a first elongate tool member having an end effector through a first sealed channel defined through an insertion tool within the access port; and
advancing a second elongate tool member having an end effector through a second sealed channel defined through the insertion tool within the access port.

21. The method of claim 20 wherein advancing the access port further comprises securing the access port to the patient.

22. The method of claim 20 wherein advancing the access port further comprises advancing the access port into the stomach with the insertion tool therein.

23. The method of claim 20 wherein advancing the access port further comprises positioning the insertion tool within a lumen defined through the access port.

24. The method of claim 21 wherein positioning the insertion tool within the lumen comprises sealing the insertion tool against the access port.

25. The method of claim 20 further comprising flexing a proximal portion of the first elongate tool member or the second elongate tool member via a flexible portion defined therealong.

26. The method of claim 20 wherein advancing the first elongate tool member further comprises performing a procedure upon at least one tissue region within the stomach with the end effector.

27. The method of claim 24 wherein the procedure performed upon the at least one tissue region is selected from the group consisting visualizing, grasping, plicating, manipulating, affixing, and securing.

28. The method of claim 20 wherein advancing the second elongate tool member further comprises performing a procedure upon at least one tissue region within the stomach with the end effector.

29. The method of claim 26 wherein the procedure performed upon the at least one tissue region is selected from the group consisting visualizing, grasping, plicating, manipulating, affixing, and securing.

30. The method of claim 20 further comprising removing at least one of the first or second elongate tool members from the insertion tool.

31. The method of claim 20 further comprising articulating the insertion tool relative to the access port.
